(19) United States
(12) Patent Application Publication
(10) Pub. No.: US 2006/0282110 A1
(43) Pub. Date: Dec. 14, 2006

(54) Advanceable, non-removable guide wire balloon catheter delivery system for a stent and method

(75) Inventors: Frank Litvack, Los Angeles, CA (US); Peter S. Brown, Palo Alto, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/485,174

(22) Filed: Jul. 11, 2006

(51) Int. Cl. A61M 29/00

(52) U.S. Cl. 606/192

(57) ABSTRACT

An advanceable, non-removable guide wire balloon catheter delivery system for a stent includes a balloon dilation catheter comprising a balloon defined by at least parts of an inner tubular element and an outer tubular element, a guidewire disposed in and having a limited range of longitudinal movement relative to the inner tubular element, and an expandable stent mounted on the balloon. A method of implanting a stent in a patient is also disclosed.

### BACKGROUND AND SUMMARY

The present invention relates generally to stent delivery systems and, more particularly, to a stent delivery system and method using an advanceable, non-removable guide wire balloon catheter delivery system.

Stent implantation procedures, particularly those for drug-eluting stents, are typically performed with predilation of a blood vessel using a balloon catheter. A guidewire is introduced into the patient's vasculature and an angioplasty balloon in a deflated condition is moved up the guidewire to the site to be dilated. When the balloon is properly positioned, the balloon is inflated to dilate the blood vessel. When the blood vessel is dilated, the balloon is deflated and withdrawn along the guidewire. A stent delivery balloon catheter is then inserted in the dilated vessel over the guidewire, the stent is expanded within the vessel, and the balloon and the guidewire are withdrawn.

A disadvantage of known stent implantation procedures is that they tend to be time consuming and involve a number of separate steps. Known over the wire and rapid exchange catheters for delivery of stents have relatively large crossing profiles or delivery diameters and it can be difficult to pass them through constricted vessels or lesion sites. Where reference is made herein to diameters of tubular members, it will be appreciated that the members may not actually be circular, particularly when compressed or otherwise under load. Frequently but not always the expression “delivery diameter” will be used herein to express a dimension of a tubular component that might not always be circular in cross-section to provide some sense of the dimensions of the component.

Another stenting procedure called direct stenting involves implantation of a stent without predilation by an angioplasty balloon. Direct stenting requires that the balloon and stent together pass through the constricted portion of the blood vessel or lesion site. When the balloon and the stent arrive at the desired location, the balloon is inflated to expand the stent. The balloon is then deflated and withdrawn, leaving the stent behind. While the direct stenting procedure can involve fewer steps and take less time than traditional stenting following angioplasty, direct stenting is generally not recommended for use with coated drug-eluting stents because the coatings can be damaged when passed through small openings.

It is desirable to provide a stent delivery system and method that can be used to implant a stent quickly and in a minimal number of steps. It is also desirable to provide a stent delivery system and method that requires a minimal crossing profile or delivery diameter such that the system and method can be used to introduce a stent to a desired location in spite of constricted blood vessel openings or tight lesions.

According to an aspect of the present invention, an advanceable, non-removable guide wire balloon catheter delivery system for a stent comprises a balloon dilation catheter comprising a balloon defined by at least parts of distal parts of an inner tubular element and an outer tubular element, a guidewire disposed in and having a limited range of longitudinal movement relative to the inner tubular element, and an expandable stent mounted on the balloon.

According to another aspect of the present invention, an advanceable, non-removable guide wire balloon catheter delivery system for a stent comprises a balloon dilation catheter comprising a balloon at a distal part thereof, the catheter being defined at least in part by at least part of a tubular element, the catheter having a proximal part of larger diameter than a reduced diameter part of a distal part of the catheter, a guidewire disposed in the tubular part and having a limited range of longitudinal movement relative to the catheter, and an expandable stent mounted on the balloon.

According to still another aspect of the present invention, a direct stenting method of implanting a stent in a patient is provided. According to the method, an advanceable, non-removable guide wire balloon catheter delivery system for a stent is provided, the system comprising a balloon dilation catheter comprising a balloon defined by at least parts of distal parts of an inner tubular element and an outer tubular element, a guidewire disposed in and having a limited range of longitudinal movement relative to the inner tubular element, and an expandable stent mounted on the balloon. The stent mounted on the balloon is passed through the patient's vasculature to a desired location, and the stent is expanded by inflating the balloon at the location.

### BRIEF DESCRIPTION OF THE DRAWINGS

The features and advantages of the present invention are well understood by reading the following detailed description in conjunction with the drawings in which like numerals indicate similar elements and in which:

FIG. 1 is a cross-sectional view of a portion of an advanceable, non-removable guide wire balloon catheter delivery system for a stent, showing a balloon in a deflated condition, according to an embodiment of the present invention;

FIG. 2 is a cross-sectional view of a portion of an advanceable, non-removable guide wire balloon catheter delivery system for a stent, showing a balloon in an inflated condition, according to an embodiment of the present invention;

FIG. 3 is a cross-sectional view of a portion of an advanceable, non-removable guide wire balloon catheter delivery system for a stent including an external insertion stop;

FIG. 4 is a cross-sectional view of a portion of an advanceable, non-removable guide wire balloon catheter delivery system for a stent according to an embodiment of the present invention; and

FIG. 5 is a cross-sectional view of a portion of an advanceable, non-removable guide wire balloon catheter delivery system for a stent according to another embodiment of the present invention.

### DETAILED DESCRIPTION

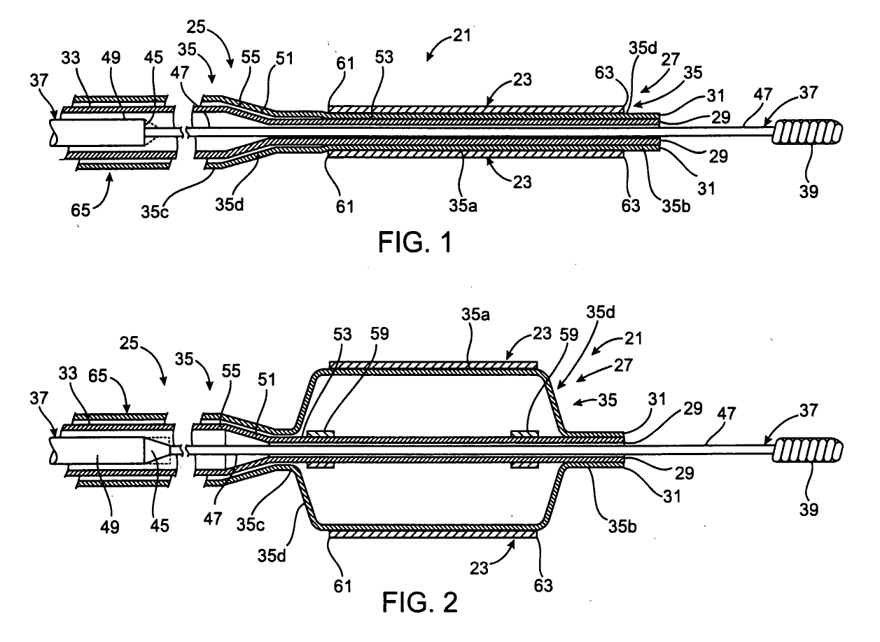

An advanceable, non-removable guide wire balloon catheter delivery system 21 for a stent 23 is shown in FIGS. 1 and 2. The system 21 comprises a balloon dilation catheter 25 comprising a balloon 27 defined by at least parts of an inner tubular element 33 and an outer tubular element 35, respectively. Distal parts 29 and 31 of the inner tubular element 33 and the outer tubular element 35 are sealed together and form part of a distal tip of the catheter 25. The outer tubular element 35 of the catheter typically includes an expandable balloon portion 35a, a distal balloon leg 35b, a proximal balloon leg 35c, and shoulder portions 35d between the expandable balloon portion and the legs. The proximal balloon leg 35c is ordinarily connected to a shaft 65 of the catheter 25, and the catheter shaft 65 is ordinarily in the form of another outer tubular member disposed outside of the inner tubular member 33 and proximal of the outer tubular member 35 forming part of the balloon 27. The catheter shaft 65 can be any suitable material, such as plastic, metal, combinations of plastic and metal, and may comprise any suitable structure, such as coils, braids, and the like. FIG. 1 shows the balloon 27 in a deflated condition while FIG. 2 shows the balloon in an inflated condition. U.S. Pat. No. 4,616,653, which is incorporated by reference, discloses a type of balloon dilation catheter with non-removable guide wire of a type generally suitable for use in connection with the balloon catheter delivery system 21. In an aspect of the invention, the delivery system 21 and stent 23 can have a small or low crossing profile or delivery diameter relative to traditional over the wire and rapid exchange catheters for delivery of stents.

The system 21 also includes a guidewire 37 disposed in and having a limited range of longitudinal movement relative to the inner tubular element 33. The guidewire 37 can be rotated relative to the inner tubular element 33. The longitudinal movability and rotatability of the guidewire 37 relative to the inner tubular element 33 facilitates accessing tight lesions and maneuvering curves or branches in a patient's vasculature. In the system 21 according to present invention, the guidewire 37 is longitudinally movable relative to the inner tubular element 33, ordinarily up to about 15 cm, although the guidewire may be movable relative to the inner tubular element over a greater or lesser distance. Typically, however, the guidewire 37 will be longitudinally movable relative to the inner tubular element 33 between about 3 cm and about 10 cm. The guidewire 37 is movable relative to the catheter 25, but is not removable. A tip 39 is typically secured to a distal part of the guidewire 37, the tip having a larger diameter than the guidewire, and is too large to be drawn through the distal end of the inner tubular element 33, thus preventing removal of the guidewire from the catheter 25. The tip 39 can be in any suitable form, such as in the form of a coil to which a semi-spherical tip is secured as disclosed in U.S. Pat. No. 4,616,653.

An expandable stent 23 is mounted on the balloon 27. The stent 23 may be a drug-eluting stent. A non-coated, drug eluting stent of the type described in WO 03/015664, which is incorporated by reference, which has drug inlays in reservoirs within the stent, is anticipated to be particularly well-suited for use in connection with the present invention because there is no coating which can be damaged or scraped off while passing the stent through small openings such as tight lesions. Other types of uncoated stents that are likely to be well-suited for use in connection with the present invention include bioresorbable drug impregnated stents and stents in which drugs are provided in channels or grooves in the stents.

It is possible to construct the system 21 in a manner that minimizes the crossing profile or delivery diameter of the stent 23 mounted on the balloon 27. For example, a distal part 47 of the guidewire 37 can be provided with at least a portion having a narrower diameter than a proximal part 49. The inner tubular element 33 and the outer tubular element 35 around the narrowed portion of the distal part 47 of the guidewire 37 can have a reduced diameter relative to portions of the inner tubular element and the outer tubular element (or catheter shaft 65 attached to outer tubular element) around larger diameter portions of the guidewire 37. As a consequence, the expandable stent 23 can have a reduced crossing profile or delivery diameter relative to systems such as those wherein a stent is implanted via a balloon catheter that is moved along a constant diameter guidewire such as over-the-wire catheterization or rapid catheter exchange. This can facilitate use of a stent in circumstances where blood vessels or lesions are too constricted to permit a stent to be implanted by conventional techniques without predilation.

Largely because of the ability of the advanceable, non-removable guide wire balloon catheter delivery system 21 for a stent 23 to have a minimal crossing profile or delivery diameter, it can be of particular use in connection with direct stenting procedures in which a stent is implanted in a patient without predilation with an angioplasty balloon. In a direct stenting procedure, the balloon 27 and stent 23 together pass through a constricted portion of a blood vessel or lesion site. When the stent 23 is positioned as desired, the balloon 27 is inflated, expanding the stent. The balloon 27 can then be deflated and the catheter system 21 can be withdrawn together with the guidewire 37, leaving the expanded stent 23 in place.

Thus, a direct stenting procedure with an advanceable, non-removable guide wire balloon catheter delivery system 21 for a stent 23 can be faster, or at least involve fewer steps, than a stenting procedure that requires, e.g., introducing, over a guidewire, a balloon catheter to the blood vessel for predilation of the blood vessel followed by removal of the balloon catheter and introduction of a stent over the same guidewire. A non-coated, drug eluting stent 23 of the type having the drug within the stent is of particular use in connection with a direct stenting procedure using the advanceable, non-removable guide wire balloon catheter delivery system 21 for a stent because a coating on the stent is not apt to be damaged or scraped off while passing the stent through small openings such as tight lesions.

In direct stenting operations, it is generally desirable to use balloons with high rated burst pressures (RBP). Typical RBPs for known balloons is about 16-18 atmospheres. The higher a balloon's rated burst pressure, the more it is likely to be able to fully expand a stent in an undilated lesion. This is important, of course, because, if the stent does not fully expand, it may be difficult to remove the balloon and it may have to be surgically removed. Also, even if the balloon can be removed when a stent has not been fully expanded, it will still be necessary to get another balloon into the stent to expand it, which can be very difficult.

Typical RBPs for known balloons is about 16-18 atmospheres. According to aspects of the present invention, the RBP of a balloon can be higher than typical RBPs while having the same or a reduced crossing profile or diameter as the known balloons at least because, according to aspects of the present invention, the reduced diameter portion 53 of the inner tubular element 31 and the reduced diameter portion 47 of the guidewire 37 permit use of an outer tubular element 35 having thicker walls than is typical, while still maintaining a small crossing profile or delivery diameter. Thicker walls in the outer tubular element can facilitate operation of balloon catheter delivery systems according to aspects of the present invention with balloons having higher RBPs, i.e., RBPs above those of typical balloon catheters, such as RBPs in the range of greater than 18 and, using presently available materials, likely up to RBPs of about 20 atmospheres or more. The higher a balloon's rated burst pressure, the more it is likely to be able to fully expand a stent in an undilated lesion. This is important, of course, because, if the stent does not fully expand, it may be difficult to remove the balloon and it may have to be surgically removed. Also, even if the balloon can be removed when a stent has not been fully expanded, it will still be necessary to get another balloon into the stent to expand it, which can be very difficult.

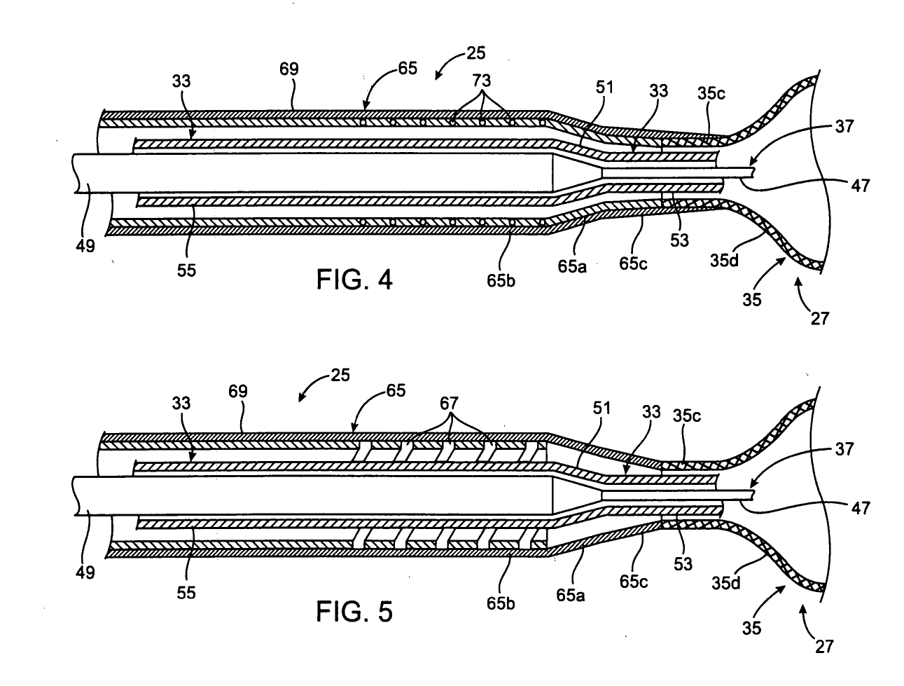

The advanceable, non-removable guide wire balloon catheter delivery system 21 for a stent 23 comprises what shall be denominated an insertion stop for limiting relative longitudinal forward movement of the guidewire 37 inside the inner tubular element 33. An embodiment of an insertion stop is seen in FIGS. 1 and 2 and comprises a guidewire transition 45 between a narrower diameter portion 47 and a larger diameter portion 49 of the guidewire 37. The illustrated embodiment of the insertion stop also comprises an inner tubular element transition 51 between a narrower diameter distal portion 53 and a larger diameter proximal portion 55 of the inner tubular element 33. As seen in FIGS. 4-5, the catheter shaft 65 may have a transition 65a from a larger diameter portion 65b to a reduced diameter portion 65c where it meets the outer tubular member 35 to facilitate keeping the crossing profile or delivery diameter of the system at a minimum.

Another form of stop that can be provided with the balloon catheter delivery system 21 prevents relative longitudinal rearward movement, i.e., retraction, of the guidewire 37 beyond a particular position relative to the balloon 27 and is herein denominated a retraction stop. In an embodiment, such a stop is provided where the proximal end of an enlarged portion of the guidewire 37, such as a proximal end of the tip 39, comes into contact with the distal ends 29 and 31 of the inner and outer tubular elements 33 and 35 or another tip of the catheter.

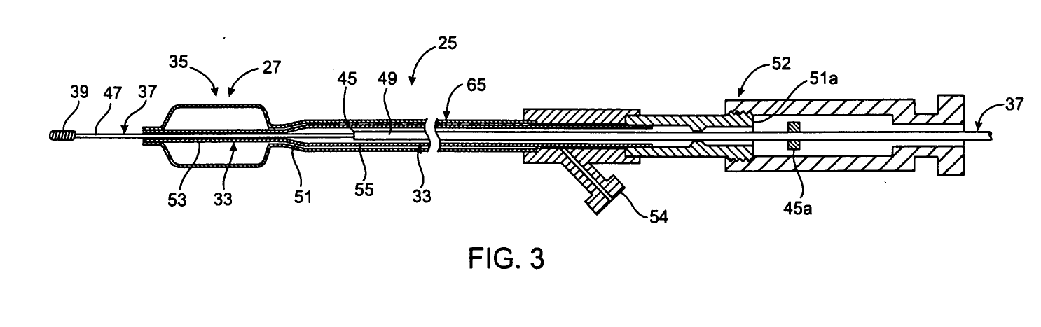

When the guidewire transition 45 contacts the inner tubular element transition 51, the guidewire 37 may be in its forwardmost position relative to the inner tubular element 33. As seen in FIG. 3, an “additional” insertion stop, hereinafter denominated as an external insertion stop, which might be in a form comprising another guidewire transition 45a and another transition 51a, can be provided in the balloon catheter delivery system 21. This “additional” or external insertion stop may be disposed in a position in which it is likely that the external insertion stop will be disposed outside of the patient's body and is therefore denominated an “external” stop, although it might also be provided so that it is likely to be disposed inside of the patient's body. A stop external to the patient's body is described here for purposes of illustration, and it will be appreciated that the references to an external stop or an external transition are not intended to limit the present invention. The additional, external transition 51a can be provided in the inner tubular element 33 or, as seen in FIG. 3, the additional, external transition 51a can be disposed proximal the inner tubular element 33, such as in an adapter 52, proximal an inflation port 54. As seen in FIGS. 1 and 2, the insertion stop can be in a position in which it will be disposed inside of the patient's body, such as near the proximal part 35c of the balloon 27. When an external insertion stop is used, it is ordinarily not an additional stop but, rather, is the only stop, and the internal reduced diameter components such as the internal guidewire transition 45 and the internal inner tubular element transition 51 forming what is denominated herein as the insertion stop will mainly serve the function of permitting a reduced diameter of the guidewire 37 and inner tube 33 under the balloon 27 so that the stent 23 crossing profile or delivery diameter dimensions can be minimized, i.e., the internal guidewire transition 45 may approach but will not ordinarily contact the inner internal tubular element transition 51. By providing the insertion stop by or before the proximal part 35c of the balloon 27, narrow diameter portions 47 and 53 of the guidewire 37 and the inner tubular element 33 can be disposed by the balloon 27 which will permit the crossing profile or delivery diameter of the balloon and stent to be minimized. Thus, the crossing profile or delivery diameter of the delivery system 21 and stent 23 can be small or low relative to known over the wire and rapid exchange catheters for the delivery of stents.

As seen in FIGS. 1-2, the guidewire transitions 45 and the inner tubular element transitions 51 can be abrupt (solid lines in FIG. 1, phantom in FIG. 2) or tapered (phantom in FIG. 1, solid lines in FIG. 2). However, it is presently expected that, ordinarily, guidewire transitions 45 and inner tubular element transition 51 will be gradual, tapered transitions providing gradual changes in the catheter's flexibility, and external transition 51a will be abrupt. The more abrupt the transition from one diameter to another, the less surface area is available to result in friction between outside surface of the guidewire transition 45a and the inside surface of the transition 51a. An abrupt transition can facilitate rotating the guidewire 37 relative to the inner tubular element 33. If an exterior insertion stop is provided, the interior transition portions will typically not be able to contact, and they may be gradual or tapered, while the exterior insertion stop will typically be abrupt to facilitate rotation of the guidewire 37 relative to the inner tubular element 33. Of course, any combination of tapered or non-tapered transitions can be used, as well. If a taper is provided to transition from a larger to a smaller diameter portion 55 and 53 of the inner tubular element transition, the taper will ordinarily extend over a length of about 0.1 cm to about 3 cm.

As seen in FIG. 2, the advanceable, non-removable guide wire balloon catheter delivery system 21 can be provided with at least one radiopaque marker 59 disposed on the inner tubular element 33 proximate at least one end 61 and/or 63 of the stent, such as to facilitate determining a location of the stent 23 inside a patient. A marker 59 can be provided at any desired location. For example, a plurality of markers 59 can be provided, such as at opposite ends of the stent 23 to facilitate precisely determining the location of the ends 61 and 63 of the stent. The marker 59 does not, however, have to be placed at either end of the stent 23.

It is desirable that the advanceable, non-removable guide wire balloon catheter delivery system 21 be capable of being pushed through small openings. U.S. patent application Ser. No. 11/251,236, filed Oct. 13, 2005, entitled RAPID EXCHANGE CATHETER WITH HYPOTUBE AND SHORT EXCHANGE LENGTH is incorporated by reference and discloses catheter systems using laser-cut hypotubes due to their excellent pushability and small diameter. The advanceable, non-removable guide wire balloon catheter delivery system 21 can include a catheter shaft 65 as seen in FIG. 5 that can comprise at least one of hypotubes or laser cut hypotubes 67 at least partially covered with a polymer sleeve which is bonded to the proximal balloon leg 35c. Instead of or in addition to hypotubes or laser cut hypotubes 67 and wall thickness variations, the catheter shaft 65 can include braids or coils 73, as seen in FIG. 4. The braids and coils may be incorporated in a polymer shaft which may optionally have a wall thickness at a proximal part 69 of the catheter shaft greater than a wall thickness of a distal part 71 of the catheter shaft.

In a method according to an aspect of the present invention, a direct stenting method of implanting a stent 23 in a patient is provided. According to the method, an advanceable, non-removable guide wire balloon catheter delivery system 21 for a stent 23 is provided, the system comprising a balloon dilation catheter 25 comprising a balloon 27 defined by at least parts of distal parts 29 and 31 of an inner tubular element 33 and an outer tubular element 35. The system 21 further comprises a guidewire 37 disposed in and having a limited range of longitudinal movement relative to the inner tubular element 33 and a full range of rotational movement. The system 21 further comprises an expandable stent 23 mounted on the balloon 27. The stent 23 mounted on the balloon 27 in a deflated condition (FIG. 1) is passed through the patient's vasculature to a desired location. The stent 23 is then expanded at the location by inflating the balloon 27.

ithout intending to limit the present invention to components having any specific dimensions, following are typical dimensions for components for use in connection with embodiments of the present invention. A stent 23 for use in connection with the present invention is typically 8-40 mm (0.31-1.57 inches) in length and has an unexpanded diameter of 1.01 mm (0.040 inches) or less, and preferably 0.094 mm (0.037 inches) or less, when fully crimped. The stent 23 typically has an expanded diameter of 2-5 mm (0.08-0.20 inches) when fully expanded by the balloon. The balloon portion 35a of the outer tubular member 35 is typically about 1-2 mm (0.04-0.08 inches) longer than the stent 23 and has a diameter of 0.63-1.0 mm (0.025-0.040 inches) when uninflated and a diameter sufficient to fully deploy the stent when inflated. The balloon portion 35a typically has a wall thickness of approximately 0.01-0.03 mm (0.0005-0.0013 inches) when deflated, although the balloon will often be folded, such as in a tri-fold, quad-fold, or more folds, for delivery, which can add to delivery diameter.

The proximal balloon leg 35c typically has an outside diameter of 0.64-0.90 mm (0.025-0.035 inches) and a wall thickness of about 0.06-0.25 mm (0.002-0.010 inches). The distal part of the catheter shaft 65 proximate the proximal balloon leg 35c can have an outside diameter of 0.066-1.02 mm (0.026-0.40 inches) where it transitions to the proximal balloon leg. This distal part of the catheter shaft 65 may step down in diameter as shown in FIGS. 4 and 5 to follow the step down in diameter to the narrower diameter portion 53 of the inner tubular element 33.

The narrow diameter portion 53 of the inner tubular element 33 can have an outside diameter of 0.03-0.58 mm (0.012-0.023 inches and a wall thickness of 0.05-0.13 mm (0.002-0.005 inches), and the larger diameter portion 55 of the inner tubular element can have an outer diameter of 0.51-0.76 mm (0.020-0.030 inches) (or about 1.5 to 2 times the diameter of the narrow diameter portion) and a wall thickness of 0.05-0.18 mm (0.002-0.007 inches). A length of the transition portion 51 of the inner tubular element 33 is typically between 0.01-3 cm (0.004-1.18 inches). The transition portion 51 of the inner tubular element 33 is typically located 1-5 cm (0.4-2 inches) from the proximal balloon leg 35c. The transition portion 51 of the inner tubular element 33 can be proximal of or at any transition 65a in the catheter shaft 65 hypotube 67. The transition portion 51 of the inner tubular element 33 is typically located within 0-5 cm (0-2 inches) of the proximal balloon leg 35c.

The transition from a large diameter portion 49 of the guidewire 37 to the narrower diameter portion 47 of the guidewire will ordinarily depend upon an amount of travel desired, although it is anticipated that it will ordinarily be between 2-15 cm (0.79-5.90 inches) behind the distal part of the guidewire, i.e., behind the tip 39. The large diameter portion 49 of the guidewire 37 typically has a diameter of about 0.35-0.45 mm (0.014-0.018 inches) and the narrow diameter portion 47 typically is about 0.15-0.254 mm (0.006-0.010 inches). The narrow diameter portion 47 generally has a diameter that is no greater than 70% of the diameter of the large diameter portion 49, and more typically no greater than 60% of the diameter of the larger diameter portion.

In the present application, the use of terms such as “including” is open-ended and is intended to have the same meaning as terms such as “comprising” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

While this invention has been illustrated and described in accordance with a preferred embodiment, it is recognized that variations and changes may be made therein without departing from the invention as set forth in the claims.

What is claimed is:

1. An advanceable, non-removable guide wire balloon catheter delivery system for a stent, comprising:
a balloon dilation catheter comprising a balloon defined by at least parts of an inner tubular element and an outer tubular element;
a guidewire disposed in and having a limited range of longitudinal movement relative to the inner tubular element; and
an expandable stent mounted on the balloon.

2. The advanceable, non-removable guide wire balloon catheter delivery system for a stent as set forth in claim 1, wherein the stent is a drug-eluting stent.

3. The advanceable, non-removable guide wire balloon catheter delivery system for a stent as set forth in claim 2, wherein the stent is a non-coated stent.

4. The advanceable, non-removable guide wire balloon catheter delivery system for a stent as set forth in claim 1, wherein a distal part of the guidewire has a narrower diameter than a proximal part.

5. The advanceable, non-removable guide wire balloon catheter delivery system for a stent as set forth in claim 1, wherein a diameter of a distal part of the guidewire is no greater than 70% of a diameter of a proximal part of the guidewire.

6. The advanceable, non-removable guide wire balloon catheter delivery system for a stent as set forth in claim 1, wherein the guidewire is longitudinally movable relative to the inner tubular element up to about 15 cm.

7. The advanceable, non-removable guide wire balloon catheter delivery system for a stent as set forth in claim 6, wherein the guidewire is longitudinally movable relative to the inner tubular element between about 3 cm and about 10 cm.

8. The advanceable, non-removable guide wire balloon catheter delivery system for a stent as set forth in claim 1, comprising a stop for limiting relative longitudinal movement of the guidewire and the inner tubular element.

9. The advanceable, non-removable guide wire balloon catheter delivery system for a stent as set forth in claim 8, wherein the stop comprises a guidewire transition between a narrower diameter portion and a larger diameter portion of the guidewire.

10. The advanceable, non-removable guide wire balloon catheter delivery system for a stent as set forth in claim 9, wherein the stop comprises an inner tubular element transition between a narrower diameter portion and a larger diameter portion of the inner tubular element.

11. The advanceable, non-removable guide wire balloon catheter delivery system for a stent as set forth in claim 9, wherein the inner tubular element transition is tapered.

12. The advanceable, non-removable guide wire balloon catheter delivery system for a stent as set forth in claim 11, wherein the inner tubular element transition is tapered over a length of about 0.1 cm to about 3 cm.

13. The advanceable, non-removable guide wire balloon catheter delivery system for a stent as set forth in claim 8, wherein the guidewire transition is tapered.

14. The advanceable, non-removable guide wire balloon catheter delivery system for a stent as set forth in claim 8, wherein the guidewire transition is non-tapered.

15. The advanceable, non-removable guide wire balloon catheter delivery system for a stent as set forth in claim 8, wherein the stop is an insertion stop adapted to limit relative longitudinal forward movement of the guidewire inside the inner tubular element.

16. The advanceable, non-removable guide wire balloon catheter delivery system for a stent as set forth in claim 15, further comprising a retraction stop adapted to limit relative longitudinal rearward movement of the guidewire inside the inner tubular element.

17. The advanceable, non-removable guide wire balloon catheter delivery system for a stent as set forth in claim 8, wherein the stop is a retraction stop adapted to limit relative longitudinal rearward movement of the guidewire inside the inner tubular element.

18. The advanceable, non-removable guide wire balloon catheter delivery system for a stent as set forth in claim 1, wherein the catheter comprises a catheter shaft comprising at least one of braids, coils, hypotubes, and laser cut hypotubes.

19. The advanceable, non-removable guide wire balloon catheter delivery system for a stent as set forth in claim 1, wherein at least a distal part of the inner tubular element has a reduced outside diameter relative to a proximal part of the inner tubular element.

20. The advanceable, non-removable guide wire balloon catheter delivery system for a stent as set forth in claim 19, wherein a diameter of at least part of the distal part of the inner tubular element is no greater than 70% of a diameter of the proximal part of the inner tubular element.

21. The advanceable, non-removable guide wire balloon catheter delivery system for a stent as set forth in claim 19, wherein at least part of a distal part of the guidewire in the distal part of the inner tubular element has a reduced outside diameter relative to a proximal part of the guidewire.

22. The advanceable, non-removable guide wire balloon catheter delivery system for a stent as set forth in claim 19, wherein the stent is mounted on the balloon over the reduced outside diameter part of the distal part of the inner tubular element.

23. The advanceable, non-removable guide wire balloon catheter delivery system for a stent as set forth in claim 1, wherein the stent comprises drug delivery reservoirs.

24. The advanceable, non-removable guide wire balloon catheter delivery system for a stent as set forth in claim 1, wherein the balloon has an RBP of greater than 18 atmospheres.

25. An advanceable, non-removable guide wire balloon catheter delivery system for a stent, comprising:
a balloon dilation catheter comprising a balloon at a distal part thereof, the catheter being defined at least in part by at least part of a tubular element, the catheter having a proximal part of larger diameter than a reduced diameter part of a distal part of the catheter;
a guidewire disposed in the tubular part and having a limited range of longitudinal movement relative to the catheter; and
an expandable stent mounted on the balloon.

26. The advanceable, non-removable guide wire balloon catheter delivery system for a stent as set forth in claim 25, wherein a distal part of the guidewire disposed in the reduced diameter part of the distal part of the catheter has a reduced diameter relative to a proximal part of the guidewire.

27. The advanceable, non-removable guide wire balloon catheter delivery system for a stent as set forth in claim 26, comprising a guidewire stop for limiting relative longitudinal movement of the guidewire and the catheter.

28. The advanceable, non-removable guide wire balloon catheter delivery system for a stent as set forth in claim 27, wherein the guidewire stop comprises a guidewire transition between the reduced diameter part of the guidewire and a larger diameter part of the guidewire.

29. The advanceable, non-removable guide wire balloon catheter delivery system for a stent as set forth in claim 28, wherein the guidewire stop comprises a catheter transition between the reduced diameter part and the larger diameter part of the catheter.

30. The advanceable, non-removable guide wire balloon catheter delivery system for a stent as set forth in claim 25, wherein the stent is a drug-eluting stent.

31. The advanceable, non-removable guide wire balloon catheter delivery system for a stent as set forth in claim 25, wherein the stent is a non-coated stent.

32. The advanceable, non-removable guide wire balloon catheter delivery system for a stent as set forth in claim 25, wherein the stent comprises drug delivery reservoirs.

33. The advanceable, non-removable guide wire balloon catheter delivery system for a stent as set forth in claim 25, wherein the balloon has an RBP of greater than 18 atmospheres.

34. A direct stenting method of implanting a stent in a patient, comprising:
providing an advanceable, non-removable guide wire balloon catheter delivery system for a stent comprising
a balloon dilation catheter comprising a balloon defined by at least parts of an inner tubular element and an outer tubular element,
a guidewire disposed in and having a limited range of longitudinal movement relative to the inner tubular element, and
an expandable stent mounted on the balloon;
passing the stent mounted on the balloon through the patient's vasculature to a desired location in a blood vessel; and
expanding the stent by inflating the balloon at the location without predilating the blood vessel.

35. The direct stenting method as set forth in claim 34, wherein at least part of a distal part of the inner tubular element has a reduced outside diameter relative to a proximal part of the inner tubular element, the stent being mounted on the balloon over the reduced outside diameter portion of the inner tubular element.

36. The direct stenting method as set forth in claim 35, wherein at least part of a distal part of the guidewire in the distal part of the inner tubular element has a reduced outside diameter relative to a proximal part of the guidewire.

37. The direct stenting method as set forth in claim 34, wherein the stent is a drug-eluting stent.

38. The direct stenting method as set forth in claim 37, wherein the stent is a non-coated stent.
